(19) United States
(12) Patent Application Publication
(10) Pub. No.: US 2008/0293814 A1
(43) Pub. Date: Nov. 27, 2008

(54) CONCENTRATE ESMOLOL

(76) Inventors: Deepak Tiwari, Raritan, NJ (US); George Owoo, North Plainfield, NJ (US); Rekha Nayak, Monmouth Junction, NJ (US); Kenneth E. Burhop, Spring Grove, IL (US)

Correspondence Address: BAXTER HEALTHCARE CORPORATION, ONE BAXTER PARKWAY, DF2-2E, DEERFIELD, IL, 60015, US

(21) Appl. No.: 11/752,037

(22) Filed: May 22, 2007

(51) Int. Cl. A61K 31/195

(52) U.S. Cl. 514/567

(57) ABSTRACT

A concentrate esmolol formulation is provided that is safer than current concentrate (e.g., 250 mg/ml) esmolol compositions. The concentrate esmolol formulation can include from about 40-60 mg/ml of esmolol hydrochloride. The concentrate esmolol composition allows a practitioner the flexibility of choosing a bolus volume for direct injection to a patient or, optionally, to use the composition to make a customized, diluted composition of esmolol. Methods of the present invention provide for the reduction of potential adverse health consequences resulting in the improper dosing of prior art concentrate compositions of esmolol. Also, a medical product is provided that includes a concentrate esmolol housed in a container, and a package housing the container and instructions.

### BACKGROUND OF THE INVENTION

The present invention is directed to improved concentrate esmolol formulations that provide for reduced risks of medication errors and are essentially free from potential injection site pain or irritation. More specifically, the invention is directed to a 40-60 mg/ml concentrate esmolol formulation preferably approved for intravenous administration that can be administered as a ready-to-use composition or diluted to desired concentrations prior to the administration to the patients.

A medication is safe and efficacious generally when administered within its proper dosage range. Administration of an improper dosage of a medication can have adverse consequences and in some cases, such dosing errors can have life threatening consequences.

There are many commonly used safe and effective liquid medications that in concentrate form could be potentially hazardous and in which the concentrate liquid is indistinguishable from a diluted form of the liquid. One widely used medication that can be provided both in concentrate, liquid form and a diluted, liquid, ready-to-use form is methyl-3-[4-(2-hydroxy-3-isopropylamino) propoxy]phenylpropionate hydrochloride (esmolol hydrochloride).

Esmolol (and its pharmaceutically acceptable salts, e.g., hydrochloride salt) and related compounds have β-adrenergic blocking activity. β-blockers are therapeutically effective agents for the treatment and prophylaxis of cardiac disorders when administered in the appropriate dosage. However, high doses can cause dangerously low cardiac output. Esmolol, which is a short-acting β-blocker, is often times used in acute care settings to control the heart rate of a patient. Ready-to-use isotonic, and concentrate formulations, of esmolol hydrochloride and related compounds are disclosed in U.S. Pat. Nos. 5,017,609, 6,310,094, and 6,528,540, incorporated herein by reference. Methods for making esmolol and related compounds and methods for treatment or prophylaxis of cardiac disorders using such compounds are disclosed in U.S. Pat. Nos. 4,387,103 and 4,593,119, incorporated herein by reference.

A current commercial esmolol concentrate formulation is available in a 10 ml solution comprising about 250 mg/ml of esmolol hydrochloride, 25% by volume ethanol, 25% by volume propylene glycol, 17 mg/ml sodium acetate trihydrate, and 0.715% by volume of glacial acetic acid. This composition is not intended for direct injection into a patient but as a stock source to be added to a larger volume diluent. Other esmolol compositions are available in the market including 10 and 20 mg/ml pre-mixed, ready-to-use solutions for infusion and 10 mg/ml vials for bolus injection. If a practitioner desires a different concentration or the use of a different diluent than as provided with the ready-to-use compositions, the practitioner can use the concentrate composition and dilute with the desired diluent and to the customized concentration.

Although the commercial, prior art concentrate and ready-to-use composition products are packaged differently, with appropriate labeling and instructions on handling, when either product, which is a clear colorless solution, is loaded into a syringe they are indistinguishable. Therefore, if the concentrate product is not diluted but mistakenly injected directly to a patient, it could lead to serious health consequences including death.

Because practitioners prefer the flexibility of using either concentrate or ready-to-use compositions of esmolol, both products are available in the hospital setting. However, since esmolol formulations are substantially clear and colorless, the concentrate formulation is visually indistinguishable from a diluted formulation. Further, both the 10 mg/ml ready-to-use composition and the 250 mg/ml concentrate composition for dilution are available in similar volumes of 10 ml each. Consequently, dosing errors can occur by the practitioners mishandling of the two compositions. Therefore, it would be desirable to provide a concentrate liquid formulation of esmolol that could mitigate the potential dosing errors described above and yet still allow the flexibility of providing a composition that could be used to make custom compositions of esmolol.

The commercial 250 mg/ml esmolol concentrate contains propylene glycol and ethanol, agents known to cause injection site pain or irritation. Therefore, it would be desirable to provide a concentrate that does not contain any propylene glycol and ethanol.

Esmolol injections are used by practitioners for rapid onset of action and generally requires dose titration based upon the body weight of the patients. For overweight patients and for fluid restrictive patients it would be highly desirable to provide a concentrated esmolol presentation that can be administered without dilution or with minimal volume dilution.

### SUMMARY OF THE INVENTION

In one aspect of the present invention, a concentrate esmolol formulation is provided. The concentrate esmolol formulation contains of from about 40-60 mg/ml of esmolol (or pharmaceutically acceptable salts thereof), and, optionally, from about 0.005 to about 2 molar (M) of a buffering agent, and pH adjusted to between about 3.5 and about 7.0.

In another aspect of the present invention a method of dosing and administering a liquid form of esmolol is provided. The method comprises the steps of providing a concentrate esmolol formulation of about 40-60 mg/ml of esmolol (or a pharmaceutically acceptable salt thereof), selecting a volume from the liquid for either direct injection to a patient or, optionally, for further dilution with a suitable diluent, followed by injection to the patient.

In another aspect of the present invention a method of mitigating substantial adverse health consequences resulting from direct dosing of concentrate esmolol formulations is provided. The method comprises the step of providing a concentrate esmolol formulation having a concentration that can be directly administered to a patient with reduced or insignificant adverse health consequences than if a similar volume of currently used concentrate esmolol compositions were likewise dosed. Also, in embodiments of the present invention wherein the volume of the presentation is about 50 ml or more, a bolus injection of the full amount would be unlikely. This provides a helpful contrast to the erroneous 10 ml bolus injections of the prior art commercial concentrate. Since normal bolus injections of a drug generally do not exceed 20 ml, a larger volume concentrate embodiment of the present invention provides the advantage of inhibiting a practitioner from the erroneous full bolus injection of such concentrate.

An advantage of the present invention is the provision of a sterile, ready-to-use, concentrate form of esmolol that can be directly infused into a patient. Such higher concentration, sterile esmolol compositions allow for the lower volume infusion to a patient, thereby reducing volumetric effects to patients with heart or other conditions sensitive to volume infusions, including those patients on fluid restriction.

Another advantage of the present invention is that, unlike prior art concentrate compositions of esmolol that contain propylene glycol and ethanol, the present invention compositions contain no irritating or harmful excipients.

Another advantage of the present invention is that it provides ready-to-use, higher concentrations of esmolol that are sterile and not subject to preparation errors that could occur with a practitioner's custom preparation of like concentrations of esmolol compositions using prior art concentrates.

### DETAILED DESCRIPTION OF THE INVENTION

The compositions of the present invention comprise esmolol, or pharmaceutically acceptable salts thereof, e.g., hydrochloride, a buffer and, optionally, an osmotic adjusting agent. As used herein, “esmolol” refers to esmolol free base and pharmaceutically acceptable salts thereof. The solution is sterile and preferably packaged in a suitable container and terminally sterilized by autoclaving. Alternatively, the sterile, esmolol concentrate can be prepared by aseptic fill procedures. The concentration of esmolol in the concentrate ranges from about 40-60 mg/ml, preferably is about 45-55 mg/ml and most preferably 50 mg/ml.

While lower concentration (e.g., 10 mg/ml) ready-to-use compositions of esmolol require an additional buffer to maintain pH, higher concentrations of esmolol in the present invention compositions, provide self-buffering capacity to the composition. Therefore, only reduced buffer is required in the compositions of the present invention. The concentrate can include a pharmaceutically acceptable buffer to aid in maintaining the pH in a range of from about 3.5 to about 7.0. Preferably, the pH is maintained between about 4.5 and about 5.5, more preferably between 4.9 and 5.1. Degradation of esmolol occurs most rapidly when the pH is outside the range of 4.0 to 6.0 and is most stable at a pH of about 5.0.

Suitable buffers are those buffers that provide sufficient buffering capacity at the desired pH range and are pharmaceutically acceptable for injection into a patient. Examples of buffers useful in the present invention include, but are not limited to, acetate, glutamate, citrate, tartrate, benzoate, lactate, gluconate, phosphate and glycine and conjugate acids thereof. The concentration of the buffer can be from about 0.005 to about 2 M. In a preferred embodiment the buffering agent comprises a combination of sodium acetate and glacial acetic acid. A preferred combination of buffers can include sodium acetate at from about 0.005 to about 0.3 M and glacial acetic acid at from about 0.05 to about 0.3 M.

In order to avoid the incidence of, or to lessen, osmotic shock (e.g., pain at the site of injection) when dosing compositions of esmolol directly to a patient without the use of a diluent, especially an osmotic adjusted diluent, it is desired to have a suitable level of osmolality contained in such direct dose compositions. Unlike the prior, ready-to-use formulations of esmolol (10 and 20 mg/ml esmolol HCl), the compositions of the present invention provide an inherent level of osmolality (about 245-400 mOsmoles/ml) without the presence of additional osmotic adjusting agents. This is due to the higher concentration of esmolol, which itself imparts a degree of osmolality to the composition. Therefore, no further osmotic adjusting agent is generally required by the compositions of the present invention. Alternatively, if desired, other suitable osmotic adjusting agents may optionally be included in the compositions of the present invention. Such agents are pharmaceutically acceptable for injection into a patient. Suitable agents include, but are not limited to, sodium chloride, dextrose, sodium bicarbonate, calcium chloride, potassium chloride, sodium lactate, and Ringers' solution. The amount of osmotic adjusting agent to be included will vary, depending on the strength of osmolality desired in the composition and other considerations including the effect the osmotic agent may have on a given patient with a given condition, e.g., the effects of sodium on a patient with congestive heart failure. Osmotic adjusting agents are typically included in the compositions of the present invention in an amount of from about 0.1 to 5 mg/ml. Preferred osmotic adjusting agents include sodium chloride and dextrose.

Suitable containers for housing the esmolol concentrate are known in the art. They include vial, syringe, bag, bottle and ampul presentations. Containers may be fabricated of polymeric materials or from glass. Preferred polymeric containers are free of polyvinylchlorine (PVC). Preferably, the container has excellent barrier properties. A preferred container retains moisture ensuring stability of the esmolol concentrate such as glass containers or polymeric containers including barrier layers or secondary packaging. An aluminum overpouch is a preferred moisture barrier for use as secondary packaging for polymeric containers lacking a moisture barrier of their own. Preferred containers should be able to withstand terminal sterilization such as autoclaving.

The compositions of the present invention are sterile. The compositions are preferably prepared and then sterilized in their final containers by autoclaving. Alternatively, the concentrate can be aseptically prepared or terminally sterilized via autoclaving separately and then placed in sterile containers using an aseptic procedure. Typical autoclave cycles used in the pharmaceutical industry to achieve terminal sterilization of the final product are 121° C. for 15 minutes. The esmolol concentrate of the present invention can be autoclaved at a temperature ranging from 115 to 130° C. for a period of time ranging from about 5 to 40 minutes with acceptable stability. Autoclaving is preferably carried out in the temperature range of about 119 to 122° C. for a period of time ranging from about 10 to 36 minutes.

In one embodiment the concentrate is housed in a clear glass or plastic syringe and terminally sterilized. These pre-filled syringes can be provided in various volumes to permit quick and easy preparation of either small volume or large volume parental dosage by dispensing the contents of the pre-filled syringes into standard pre-filled intravenous fluid bags or, optionally, directly dosed to a patient.

In another embodiment of the present invention, a medical product includes a container housing an esmolol concentrate and instructions kept together in a single package. The instructions can inform the practitioner that, depending on the desired dose and patient information and condition, whether to use the composition as an undiluted, ready-to-use injection or to further dilute with a desired diluent.

The compositions of the present invention provide the flexibility of providing a composition useful as a ready-to-use composition or as a composition useful for further dilution. As a ready-to-use presentation, this high concentration composition can be administered to patients requiring rapid onset of action, and also to overweight patients. Furthermore, as this composition contains a higher concentration of esmolol, smaller volumes of infusion can be administered to patients under fluid restriction. Table 1 shows reduction of infusion rate based on the concentration of esmolol injection used.

TABLE 1

| | Dose required by | Concentration of | | |
|---|---|---|---|---|
| | Patients weighing | Esmolol Injection | | |
| Dose | 75 Kg | To be used | | Rate of Infusion |
| 300 μg/Kg/min | 22500 μg/min | 10 | mg/ml | 2250 μL/min |
| | | 20 | mg/mL | 1125 μL/min |
| | | 50 | mg/mL | 450 μL/min |
| 200 μg/kg/min | 15000 μg/min | 10 | mg/mL | 1500 μL/min |
| | | 20 | mg/mL | 750 μL/min |
| | | 50 | mg/mL | 300 μL/min |

If a practitioner desires a lower concentration of esmolol and/or a preferred diluent to infuse into the patient in conjunction with esmolol dosing, the practitioner may desire to dilute the compositions of the present invention. Suitable diluents include diluents used by practitioners skilled in the art. Typical examples include, sodium chloride, Ringers' and dextrose solutions. While the desired, diluted concentration of esmolol will vary, typical concentrations range from about 1 to about 25 mg/ml, and preferably 10 mg/ml.

Suitable routes for parenteral administration include intravenous, subcutaneous, intradermal, intramuscular, intraarticular and intrathecal. The diluted concentrate is preferably administered by intravenous infusion.

The following example compositions and method of manufacture further illustrate the invention but should not be construed as limiting its scope.

### EXAMPLE 1

The following describes the preparation of esmolol compositions of the present invention. The concentration of each ingredient of the compositions are provided in Tables 1 and 2 as follows:

TABLE 1
Formulations 1-3

| Ingredients | Formulation 1 | Formulation 2 | Formulation 3 |
|---|---|---|---|
| Esmolol HCl | 50 mg/mL | 50 mg/mL | 50 mg/mL |
| Sodium Acetate | — | 1.4 mg/mL | 0.7 mg/mL |
| Trihydrate, USP | | | |
| Glacial Acetic | 0.546 mg/mL | 0.546 mg/mL | 0.546 mg/mL |
| Acid, USP | | | |
| Sodium Chloride, | 1 mg/mL | 1 mg/mL | 1 mg/mL |
| USP | | | |
| Water for injection | qs | qs | qs |

TABLE 2
Formulations 4-6

| Ingredients | Formulation 4 | Formulation 5 | Formulation 6 |
|---|---|---|---|
| Esmolol HCl | 50 mg/mL | 50 mg/mL | 50 mg/mL |
| Sodium Acetate | 1.4 mg/mL | 2.8 mg/mL | 2.8 mg/mL |
| Trihydrate, USP | | | |
| Glacial Acetic | 0.546 mg/mL | 0.546 mg/mL | 0.546 mg/mL |
| Acid, USP | | | |
| Sodium Chloride, | — | 1 mg/mL | — |
| USP | | | |
| Dextrose, USP | 1 mg/mL | — | — |
| Water for injection | qs | qs | qs |

In the foregoing Formulations 1-6, the pH may be adjusted to a range of from 4.5-5.5, and preferably 5.0. The equipment and glassware for compounding, filtering, and filling are properly washed and depyrogenated. The filter assembly, filling tube assembly, and other parts and equipment are sterilized.

Eighty percent (80%) of the final volume of cool water for injection is collected in a compounding tank. Glacial acetic acid and, optionally, sodium acetate are then added to the tank. Esmolol Hydrochloride is weighed and added to the tank. Optionally, sodium chloride or dextrose is then weighed and added to the tank. The solution is stirred until all excipients are dissolved. The solution is then adjusted to pH 5.0 with 1.0N sodium hydroxide or hydrochloric acid. The solution is brought to final volume with water for injection and mixed. The esmolol concentrate is transferred to a container and autoclaved to provide an esmolol hydrochloride solution having a concentration of about 50 mg/ml.

Although the present invention has been described by reference to certain preferred embodiments, it should be understood that the preferred embodiments are merely illustrative of the principles of the present invention. Therefore, modifications and/or changes may be made by those skilled in the art without departing from the true spirit and scope of the invention as defined by the appended claims.

1. A concentrate esmolol composition comprising:
a) about 40-60 mg/ml of esmolol hydrochloride; and
b) from about 0.01 to about 2 M of a buffering agent;
wherein the composition has a pH of about 4.0 to 6.0.

2. The composition of claim 1, wherein the buffering agent comprises at least one of acetate, glutamate, citrate, tartrate, benzoate, lactate, gluconate, phosphate and glycine and conjugate acids thereof.

3. The composition of claim 2, wherein the buffering agent comprises sodium acetate and acetic acid.

4. The composition of claim 1, further comprising an osmotic adjusting agent.

5. The composition of claim 4, wherein the osmotic adjusting agent is selected from the group consisting of dextrose, sodium chloride, sodium bicarbonate, calcium chloride, potassium chloride, sodium lactate and Ringer's solution.

6. The composition of claim 4, wherein the osmotic adjusting agent is present in an amount of from about 0.1 to about 5 mg/mL.

7. The composition of claim 4 comprising:
a) about 45 to 55 mg/mL esmolol HCl;
b) about 0.01 M glacial acetic acid; and
c) about 1 mg/ml sodium chloride.

8. The composition of claim 4 comprising:
a) about 45 to 55 mg/mL esmolol HCl;
b) about 0.01 M sodium acetate;
c) about 0.01 M glacial acetic acid; and
d) about 1 mg/mL sodium chloride.

9. The composition of claim 4 comprising:
a) about 45 to 55 mg/mL esmolol HCl;
b) about 0.005 M sodium acetate;
c) about 0.01 M glacial acetic acid; and
d) about 1 mg/mL sodium chloride.

10. The composition of claim 4 comprising:
a) about 45 to 55 mg/mL esmolol HCl;
b) about 0.01 M sodium acetate;
c) about 0.01 M glacial acetic acid; and
d) about 1 mg/mL dextrose.

11. The composition of claim 4 comprising:
a) about 45 to 55 mg/mL esmolol HCl;
b) about 0.02 M sodium acetate;
c) about 0.01 M glacial acetic acid; and
d) about 1 mg/mL sodium chloride.

12. The composition of claim 1 comprising:
a) about 45 to 55 mg/mL esmolol HCl;
b) about 0.02 M sodium acetate; and
c) about 0.01 M glacial acetic acid.

13. A medical product comprising:
a) a composition comprising from about 40 to about 60 mg/ml of esmolol hydrochloride and from about 0.01 to about 2 M of a buffering agent housed in a container;
b) instructions directing a practitioner to use the composition for direct injection or for dilution and then injection; and
c) a package housing the container and instructions.

14. The medical product of claim 13, wherein the buffering agent comprises at least one of acetate, glutamate, citrate, tartrate, benzoate, lactate, gluconate, phosphate and glycine and conjugate acids thereof.

15. The medical product of claim 13, wherein the buffering agent comprises sodium acetate and acetic acid.

16. The medical product of claim 13, wherein the concentrate esmolol formulation further comprises an osmotic adjusting agent

17. The product of claim 16 wherein the composition comprises:
a) about 45 to 55 mg/mL esmolol HCl;
b) about 0.01 M glacial acetic acid; and
c) about 1 mg/ml sodium chloride.

18. The product of claim 16 wherein the composition comprises:
a) about 45 to 55 mg/mL esmolol HCl;
b) about 0.01 M sodium acetate;
c) about 0.01 M glacial acetic acid; and
d) about 1 mg/mL sodium chloride.

19. The product of claim 16 wherein the composition comprises:
a) about 45 to 55 mg/mL esmolol HCl;
b) about 0.005 M sodium acetate;
c) about 0.01 M glacial acetic acid; and
d) about 1 mg/mL sodium chloride.

20. The product of claim 16 wherein the composition comprises:
a) about 45 to 55 mg/mL esmolol HCl;
b) about 0.01 M sodium acetate;
c) about 0.01 M glacial acetic acid; and
d) about 1 mg/mL dextrose.

21. The product of claim 16 wherein the composition comprises:
a) about 45 to 55 mg/mL esmolol HCl;
b) about 0.02 M sodium acetate;
c) about 0.01 M glacial acetic acid; and
d) about 1 mg/mL sodium chloride.

22. The product of claim 13 wherein the composition comprises:
a) about 45 to 55 mg/mL esmolol HCl;
b) about 0.02 M sodium acetate; and
c) about 0.01 M glacial acetic acid.

23. A method of providing a reduction in the potential for substantial adverse health consequences resulting from an improper dosing of an esmolol concentrate liquid composition comprising the steps of:
a) providing a sterile, liquid composition comprising about 40-60 mg/ml of esmolol hydrochloride; and
b) from about 0.01 to about 2 M of a buffering agent;
wherein the composition has a pH of about 4.0 to 6.0.

24. The method of claim 23, wherein the buffering agent comprises at least one of acetate, glutamate, citrate, tartrate, benzoate, lactate, gluconate, phosphate and glycine and conjugate acids thereof.

25. The method of claim 23, wherein the buffering agent comprises sodium acetate and acetic acid.

26. The method of claim 23, further comprising an osmotic adjusting agent.

27. The method of claim 23, wherein the osmotic adjusting agent is selected from the group consisting of dextrose, sodium chloride, sodium bicarbonate, calcium chloride, potassium chloride, sodium lactate and Ringer's solution.

28. The method of claim 23, wherein the osmotic adjusting agent is present in an amount of from about 0.1 to about 5 mg/mL.

29. The method of claim 27, wherein sterile, liquid composition comprises:
a) about 45 to 55 mg/mL esmolol HCl;
b) about 0.01 M glacial acetic acid; and
c) about 1 mg/ml sodium chloride.

30. The method of claim 27, wherein sterile, liquid composition comprises:
a) about 45 to 55 mg/mL esmolol HCl;
b) about 0.01 M sodium acetate;
c) about 0.01 M glacial acetic acid; and
d) about 1 mg/mL sodium chloride.

31. The method of claim 27, wherein sterile, liquid composition comprises:
a) about 45 to 55 mg/mL esmolol HCl;
b) about 0.005 M sodium acetate;
c) about 0.01 M glacial acetic acid; and
d) about 1 mg/mL sodium chloride.

32. The method of claim 27, wherein sterile, liquid composition comprises:
a) about 45 to 55 mg/mL esmolol HCl;
b) about 0.01 M sodium acetate;
c) about 0.01 M glacial acetic acid; and
d) about 1 mg/mL dextrose.

33. The method of claim 27, wherein sterile, liquid composition comprises:
a) about 45 to 55 mg/mL esmolol HCl;
b) about 0.02 M sodium acetate;
c) about 0.01 M glacial acetic acid; and
d) about 1 mg/mL sodium chloride.

34. The method of claim 23, wherein sterile, liquid composition comprises:
a) about 45 to 55 mg/mL esmolol HCl;
b) about 0.02 M sodium acetate; and
c) about 0.01 M glacial acetic acid.
